(19) United States
(12) Patent Application Publication
(10) Pub. No.: US 2014/0276823 A1
(43) Pub. Date: Sep. 18, 2014

(54) Interchangeable Pin Holder System and Method

(71) Applicant: pro med instruments GmbH, Freiburg im Breisgau (DE)

(72) Inventor: Matthias E. Schuele, Freiburg (DE)

(73) Assignee: pro med instruments GmbH, Freiburg im Breisgau (DE)

(21) Appl. No.: 14/205,931

(22) Filed: Mar. 12, 2014

Related U.S. Application Data
(60) Provisional application No. 61/778,321, filed on Mar. 12, 2013.

(51) Int. Cl. A61B 17/60

(52) U.S. Cl. CPC A61B 17/60
USPC 606/59

(57) ABSTRACT

A stabilization device for use in the medical field includes an interchangeable pin holder assembly. The pin holder assembly has a first portion that is separable from a second portion such that the first portion can be replaced with a similar first portion having one or more components of a different size to accommodate patients of different sizes or to accommodate different stabilization techniques. A method for interchanging a portion of a pin holder assembly includes aligning components of the pin holder assembly and applying force in either a distal or proximal direction depending on whether or not the interchangeable portion of the pin holder assembly is being removed or attached.

### PRIORITY

This application claims priority to U.S. Provisional Patent Application Ser. No. 61/778,321, filed Mar. 12, 2013, entitled “Interchangeable Pin Holder System and Method,” the disclosure of which is incorporated by reference herein.

### BACKGROUND

In the medical field, certain procedures use stabilization devices to stabilize a patient or portion of a patient during the procedure. With procedures where it is desirable to stabilize the patient's head and/or neck, various kinds of head fixation devices (HFDs) can be used. One such HFD is a skull clamp that preferably uses a three or more point fixation using pins that contact the patient's skull. These pins can be selectively retained in a pin holder assembly or system that makes up a component of the HFD.

While a variety of HFDs and pin holder assemblies have been made and used, it is believed that no one prior to the inventor(s) has made or used an invention as described herein.

### SUMMARY

A stabilization device for use in the medical field includes an interchangeable pin holder assembly. The pin holder assembly has a first portion that is separable from a second portion such that the first portion can be replaced with a similar first portion having one or more components of a different size to accommodate patients of different sizes or to accommodate different stabilization techniques.

A method for interchanging a portion of a pin holder assembly includes aligning components of the pin holder assembly and applying force in either a distal or proximal direction depending on whether or not the interchangeable portion of the pin holder assembly is being removed or attached.

In some versions of the device, an interchangeable pin holder assembly for use with a stabilization device includes a first member comprising a groove and a second member comprising at least one resiliently biased member. The at least one resiliently biased member is configured to selectively engage with the groove to inhibit unintended separation of the first member from the second member when the at least one resiliently biased member is positioned within the groove. The at least one resiliently biased member also provides a level of feedback, e.g., tactile or audible, when attaching components of the interchangeable pin holder assembly. The pin holder assembly also includes a first rotation limiting member and a second rotation limiting member. The first rotation limiting member is configured to engage with the second rotation limiting member to prevent rotation of the first member relative to the second member.

In some versions of the methods for interchanging a portion of a pin holder assembly of a stabilization device, the method includes applying a force on a first removable portion of the pin holder assembly in a direction away from a fixed portion of the pin holder assembly, causing at least one resilient member to disengage a recessed area, and causing a first rotational lock member to disengage from a second rotational lock member. Further method steps can include aligning a second removable portion of the pin holder assembly with the fixed portion of the pin holder assembly, and applying a force on the second removable portion in a direction toward the fixed portion of the pin holder assembly.

In some versions of the device, an interchangeable pin holder assembly for use with a stabilization device includes a first member and a second member that are configured to eliminate either or both lateral movement and vertical movement between a first portion of the interchangeable pin holder assembly and a second portion of the interchangeable pin holder assembly when the first portion is selectively connected with the second portion. Also, the interchangeable pin holder assembly includes a third member and a fourth member that are independent from the first member and the second member, with the third and fourth members configured to eliminate rotational movement between the first portion of the interchangeable pin holder assembly and the second portion of the interchangeable pin holder assembly when the first portion is selectively connected with the second portion.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements.

FIG. 1 depicts a perspective view of an exemplary HFD in the form of a skull clamp including an exemplary interchangeable pin holder assembly.

FIG. 2 depicts a side view of an upper portion of the skull clamp of FIG. 1.

FIG. 3 depicts a side view of the upper portion of the skull clamp of FIG. 1, showing a portion of the interchangeable pin holder assembly detached from the remainder of the pin holder assembly.

FIG. 4 depicts a partially exploded perspective view of the pin holder assembly of FIG. 1.

FIG. 5 depicts another partially exploded perspective view of the pin holder assembly of FIG. 1.

FIG. 6 depicts a partial cross section view of the upper portion of the skull clamp as similarly shown in FIG. 2.

FIG. 7 depicts a partial cross section view of the upper portion of the skull clamp as similarly shown in FIG. 3.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

FIG. 1 illustrates an exemplary HFD in the form of skull clamp (10). Skull clamp (10) comprises first arm (12) and second arm (14) that can adjustably connect to accommodate a range of sizes of patients. An upper portion of each arm (12, 14) comprises an opening sized to receive a pin holder assembly. In the present example, skull clamp (10) comprises single pin holder assembly (100) near the upper portion of first arm (12) and dual pin holder assembly (200) near the upper portion of second arm (14). Pin holder assemblies (100, 200) are configured to selectively retain pins (not shown) that are configured to engage the skull of a patient when skull clamp (10) is in use to stabilize at least a portion of the patient. In the present example, pin holder assembly (200) is configured as an interchangeable pin holder assembly that allows for a portion of pin holder assembly (200) to be removed and replaced with a similar portion of pin holder assembly (200) having a larger or smaller size to accommodate patients having different head sizes.

Pin holder assembly (200) is shown as being attached to arch member (202). Arch member (202) comprises first and second ends (210, 212) that are each configured to receive and selectively retain a pin (not shown). Arch member (202) attaches to pin holder assembly (200) in the present example by way of threaded bolt (214) engaging a threaded nut. Arch member (202) is operable to adjustably rotate about threaded bolt (214).

FIG. 2 illustrates a side view of interchangeable pin holder assembly (200) of skull clamp (10). Pin holder assembly (200) comprises connector (204), adapter (206), and actuator (208). As shown in FIG. 1, connector (204) attaches arch member (202) to pin holder assembly (200). In FIG. 2, arch member (202) has been removed to show attachment hole (205), which permits the connection between connector (204) and arch member (202). Connector (204) is also configured to attach with adapter (206) as will be described in greater detail below. Adapter (206) is configured to attach with actuator (208) as will be described in greater detail below. As will also be described in greater detail below, actuator (208) is operable to selectively lock and unlock the rotational position of pin holder assembly (200) such that pin holder assembly (200) is adjustable to any of a number of desirable positions relative to a patient's head.

FIG. 3 illustrates another side view of pin holder assembly (200), showing first portion (216) detached from second portion (218) of pin holder assembly (200). In the illustrated version, connector (204) comprises at least one protruding member (220) that extends away from body portion (222) of connector (204) toward adapter (206). Also in the illustrated version, adapter (206) comprises extension (224) that includes groove (226). On the distal side of groove (226) (the distal side being the side that is nearest connector (204)), extension (224) comprises first and second sloped ridges (228, 230) or chamfered edges. On the proximal side of groove (226) (the proximal side being the side that is nearest actuator (208)), extension (224) comprises flange (232). More detail and description of these and other components and features will be described below and explain how first portion (216) selectively engages second portion (218) such that pin holder assembly (200) includes interchangeability.

FIGS. 4 and 5 illustrate partially exploded perspective views of pin holder assembly (200) and second arm (14). As shown in these illustrated versions, in addition to protruding member (220) and body portion (222), connector (204) comprises first wing member (234), second wing member (236), and slot (238) located between wing members (234, 236). Within body portion (222) are first and second threaded openings or bores (240, 242) located in the top and bottom areas of body portion (222) as shown, and third opening or bore (244) located in proximal surface (246) of body portion (222). For the purpose of this disclosure, openings and bores shall be considered interchangeable terms used to describe a hole in a structure. First and second threaded pressure pins (248, 250) of connector (204) are configured to threadably engage first and second threaded openings (240, 242). First and second threaded pressure pins (248, 250) each comprise resilient spherical portions (252) on one end and slots (254) on the opposite end. Third opening (244) is configured to receive extension (224) of adapter (206) when connector (204) is attached to adapter (206) as will be described in greater detail below.

Still referring to FIGS. 4-5, in addition to extension (224), adapter (206) comprises washer (256), nut (258), and cap (260). Extension (224) comprises threaded outer opening (262) and threaded inner opening (264). When assembled washer (256) is positioned against annular surface (266) within outer opening (262), followed by nut (258) and cap (260). Also when assembled, nut (258) threadably engages distal portion (267) of threaded rod (268) of actuator (208). Cap (260) comprises threads (261) that engage threaded outer opening (262) to retain cap (260) within adapter (206). Cap (260) also includes slot (263) to assist in engaging cap (260) with adapter (206). Adapter (206) further comprises recess (270) that is configured to receive protruding member (220) of connector (204) when pin holder assembly (200) is fully assembled. Threaded inner opening (264) of adapter (206) is configured to engage threaded rod (268) of actuator (208) also, and when assembled this engagement is with a more central portion of threaded rod (268) as will be shown and described further below.

As can best be seen in FIG. 6, adapter (206) also comprises starburst member (272) on its proximal facing surface. Starburst member (272) is configured to selectively engage with complementary starburst member (not shown) of actuator (208) when pin holder assembly (200) is fully assembled. In the present example, starburst member (272) is a separate component that attaches to the proximal facing surface of adapter (206). In other versions starburst member (272) can be unitary with the remainder of adapter (206). In the illustrated version, opening of second arm (14) has a general star-shape, and starburst member (not shown) of actuator (208) has a complementary star-shape such that it fits within the opening of second arm (14). With these complementary star-shapes, starburst member (not shown) of actuator (208) is prohibited from rotating within the opening of second arm (14). However, starburst member (not shown) of actuator (208) can translate relative to the opening in second arm (14) to engage and disengage starburst member (272) of adapter (206) as will be described further below.

FIG. 6 illustrates a section view of pin holder assembly (200) in its assembled state, while FIG. 7 illustrates a section view of pin holder assembly (200) with first portion (216) detached from second portion (218). In its assembled state, third opening (244) of connector (204) receives extension (224) of adapter (206). First and second pressure pins (248, 250) of connector (204) engage groove (226) of extension (224) of adapter (206). First and second pressure pin (248, 250) are resiliently biased with sphere portions (252) such that sphere portions (252) extend toward and seat within groove (226) thereby attaching connector (204) with adapter (206) as shown. In the present example first and second pressure pins (248, 250) comprise cavities (276). Cavities (276) contain springs (278) that create the resilient bias for sphere portions (252). In another version, cavities (276) are sufficiently sealed by sphere portions (252) and cavities (276) contain a compressible fluid that creates the resilient bias for sphere portions (252). Still other ways to create the resilient bias for sphere portions (252) will be apparent to those of ordinary skill in the art in view of the teachings herein.

In addition to first and second pressure pins (248, 250) engaging groove (226) of adapter (206), protruding member (220) of connector (204) seats within recess (270) of adapter (206). With this engagement configuration, connector (204) is securely yet selectively attached to adapter (206) in a way where connector (204) does not rotate independent from adapter (206) but in unison with any rotation of adapter (206). In some versions, more than one protruding member (220) and recess (270) are used to prohibit relative rotation of connector (204) with respect to adapter (206). In the present example, protruding member (220) is generally considered a male connecting structure and recess (270) is generally considered a female connecting structure. In some versions, protruding member (220) and recess (270) can be replaced with any other suitable male to female connecting structures. Still in other versions, protruding member (220) can be located on adapter (206) while recess (270) can be located on connector (204). In view of the teachings herein, other ways to attach connector (204) and adapter (206) will be apparent to those of ordinary skill in the art.

In the illustrated version, first portion (216) is connectable to second portion (218) in a way that there is preferably no play or free movement, either rotationally, laterally, or vertically, between first portion (216) and second portion (218) when they are connected. That is, once connected there is preferably no movement, or less preferably very little movement, between first portion (216) and second portion (218). Thus once connected the first portion (216) and second portion (218) have a tight connection such that they may feel as if they are a solid and single component. To achieve the tight connection with preferably no play or movement, or less preferably very little play or movement, between connected first portion (216) and second portion (218), tight tolerances are used when fabricating the structures or components that limit free movement either rotationally, laterally, or vertically.

To achieve no play or free movement rotationally, tight tolerances are used when fabricating protruding member (220) and recess (270). In this fashion, the width of protruding member (220) is matched or substantially matched with the width of recess (270). In some versions, while a tight tolerance is used in the width dimension as mentioned, it is not necessary or required to have a tight tolerance or match in the length dimension between recess (270) and protruding member (220). With the tight tolerance in the width dimension, when connector (204) is attached with adapter (206), the abutment or contact between the sides of protruding member (220) and the sides of recess (270) provide no play or free movement rotationally between the components. As understood from the illustrated version, rotational movement is considered that rotational movement about an axis defined by the length of threaded rod (268).

To achieve no play or free movement laterally and vertically, tight tolerances are used when fabricating extension (224) of adapter (206) and third opening (244) of connector (204). In this fashion, the diameter of third opening (244) is matched or substantially matched with the diameter of extension (224). Therefore, when connector (204) is attached with adapter (206), the abutment or contact between the sides of extension (224) and the sidewalls of third opening (244) provide no play or free movement laterally or vertically (or any diagonal direction between lateral and vertical for that matter) between the components. As understood from the illustrated version, lateral movement is considered that linear movement along an axis that would be parallel to an axis extending into and out of the page of the views of FIGS. 2 and 3. Lateral movement can be considered movement in the X-direction. As also understood from the illustrated version, vertical movement is considered that linear movement along an axis that would be parallel with a longitudinal axis defined by the upright vertical portion of arm (14). Vertical movement can be considered movement in the Y-direction. Also, lateral and vertical movement (or movement in the X and Y directions respectively) are perpendicular to movement in the Z-direction described in the following paragraph. These directions of movement are also depicted by respective axes in FIG. 1.

Free movement of first portion (216) relative to second portion (218) is further limited in the Z-direction, which can be considered the direction along an axis defined by the length of threaded rod (268), which would be the same as the direction in which first portion (216) is moved relative to second portion (218) when connecting first portion (216) to second portion (218) or removing first portion (216) from second portion (218). To achieve no or limited free movement in the Z-direction, tight tolerances are used when fabricating bores (240, 242) of connector (204) relative to the position of groove (226) of adapter (206). For example, in some versions bores (240, 242) are positioned such that when connector (204) is attached to adapter (206), bores (240, 242) are centered over groove (226).

Furthermore, the width of groove (226) and the diameter of sphere portions (252) of pressure pins (248, 250) are such that when sphere portions (252) reside within groove (226), sphere portions (252) fill the width of groove (226) such that on one side sphere portions (252) contacts first sloped ridge (228) and on an opposite side sphere portions (252) contacts sidewall (231) of flange (232). In some versions, such as the illustrated version shown in FIG. 6, sphere portions (252) substantially fill the width of groove (226) when sphere portions (252) reside within groove (226), and thus sphere portions (252) are not required completely fill the width of groove (226) and thus not required to make and maintain simultaneous contact with first sloped ridge (228) and sidewall (231) of flange (232) in all versions. Even so, in such versions where sphere portions (252) substantially fill the width of groove (226), no play or movement of first portion (216) relative to second portion (218) in the Z-direction is achieved by the abutment of sphere portions (252) with first sloped ridge (228) on one side, and the abutment of the end of protruding member (220) with the wall of recess (270) on the other side. Thus in the illustrated version of FIG. 6, these two abutments prevent there being play or free movement between connector (204) and adapter (206) in the Z-direction. In some versions, some small amount of play or free movement may be permitted in the Z-direction when a patient's head is not positioned tightly within skull clamp (10). However, when a patent's head is positioned within skull clamp (10), and skull clamp (10) tightened, any movement in the Z-direction ceases as the contact between pin holder assemblies (100, 200) and the patient's head is sufficient to prevent any movement of pin holder assemblies (100, 200) in the Z-direction.

In the illustrated version, the structures for preventing play or free movement rotationally are separate or independent from the structures for preventing play or free movement laterally and vertically. In other words, the structures that limit rotational movement are separate and independent from the structures that limit non-rotational movement such as movement laterally and vertically. Therefore, when making pin holder assembly (200) it is possible to fabricate the structures that limit the motion rotationally with very tight tolerances, and at the same time fabricate the structures that limit the motion laterally and vertically with very tight tolerances. But at the same time, it is not necessary or required to have very tight tolerances between these two structures that limit motion in different directions. Again, in other words, at the same time it is not necessary or required to have tight tolerances between the structures used to limit rotational movement and the structures used to limit non-rotational movement (i.e. movement laterally and/or vertically or in any direction along a plane parallel with proximal surface (246) of connector (204)). For example, while tight tolerances would exist between the widths of protruding member (220) and recess (270), and similarly tight tolerances would exist between the diameter of extension (224) and third opening (244), tight tolerances would not be necessary for the distance or spacing between, e.g., extension (224) and recess (270), or e.g., third opening (244) and protruding member (220). Of course tight tolerances may still be used for the relative dimensions between these components nonetheless if desired.

In this respect, tolerance refers to controlling the relative spacing between components, with a tight tolerance meaning that the spacing is precise or important with very little to no variance permitted during fabrication. A tolerance that is not a tight tolerance, on the contrary, would allow the spacing between the components to be imprecise or not important and some amount of variance would be permitted during fabrication. Furthermore, the use of tight tolerances between certain components described herein controls restrictions to movement in certain directions. Similarly a lack of tight tolerances between certain components described herein would not be considered controlling as to the restrictions on movement. By way of example and not limitation, while there are tight tolerances used between recess (270) and protruding member (220), and separately between extension (224) and third opening (244), it is not required to have a tight tolerance between protruding member (220) and opening (244). In other words, the distance or spacing between protruding member (220) and opening (244) is not important when fabricating first portion (216) and controlling restrictions on movement between first portion (216) and second portion (218). Similarly, it is not required to have a tight tolerance between recess (270) and extension (224). In other words, the distance or spacing between recess (270) and extension (224) is not important when fabricating second portion (218) and controlling restrictions on movement between first portion (216) and second portion (218).

Also, in some versions the structures for preventing play or free movement in the Z-direction are separate or independent from the structures for preventing play or free movement rotationally. For instance, in versions where sphere portions (252) fill the width of groove (226) and where the width of protruding member (220) match the width of recess (270), the structures that prevent play or free movement in the Z-direction and rotationally are separate or independent from one another. In such versions, tight tolerances are used for each structure as mentioned above, but at the same time tight tolerances between these structures themselves are not required as they are independent in their structure and the functions each accomplish. In view of the teachings herein, other ways, structures, and configurations to prevent play or free movement between first portion (216) and second portion (218) when connecting these components will be apparent to those of ordinary skill in the art.

As mentioned already, in some circumstances it is desirable to replace first portion (216) of pin holder assembly (200) with another similar first portion of pin holder assembly having a different size. For example, when using skull clamp (10) on an adult patient first and then later switching to use skull clamp (10) on a pediatric patient, it may be desired to replace first portion (216) to another similar first portion having a smaller arch member (202), in particular one that is configured with a greater curvature to better align with what would be a smaller circumference of a pediatric patient's head. In such instances, first portion (216) can have all the same component parts with the exception of the different sized arch member (202).

To replace or interchange first portion (216), first portion (216) is grasped and pulled in the distal direction such that force is applied on first portion (216) in the distal direction. In one example a user can grasp first portion (216) around arch member (202). With sufficient force exerted on first portion (216) in the distal direction, resilient sphere portions (252) contact first sloped ridge (228), and due to the distal force applied by the user, first sloped ridge (228) effectively exerts force on resilient sphere portions (252) to partially displace them within cavities (276). In this example, the force effectively exerted by first sloped ridge (228) is generally perpendicular to the force applied on first portion (216) in the distal direction. With resilient sphere portions (252) at least partially displaced within cavities by first sloped ridge (228), connector (204) is then able to separate from adapter (208) such that first portion (216) is separated from second portion (218) of pin holder assembly (200). With this detaching movement, protruding member (220) of connector (204) is moved in the distal direction away from and out of recess (270) of adapter (206).

To install a replacement first portion (216) after the prior first portion (216) has been removed, the process is generally reversed. A user grasps the replacement first portion (216) and aligns third opening (244) with extension (224) of adapter (206). Next the user pushes first portion (216) in the proximal direction toward the adapter (206). Extension (224) aligns with and fits within third opening (244) of connector (204). Before extension (224) is positioned within third opening (244), resilient sphere portions (252) are protruding slightly into the space defined by third opening (244). As extension (224) is positioned within third opening (244), second sloped ridge (230) contacts resilient sphere portions (252) effectively exerting force on resilient sphere portions (252) to partially displace them within cavities (276). This allows extension (224) to fully be positioned within third opening (244) such that first and second pressure pins (248, 250) ultimately seat within groove (226) of extension (224). At or about the same time, protruding member (220) is aligned with recess (270) and is seated within recess (270). Because of the cylindrical shape of extension (224), first portion (216) can be rotated to align protruding member (220) with recess (270) while extension (224) is positioned at least partially within third opening (244). With protruding member (220) fully seated within recess (270) and with first and second pressure pins (248, 250) full seated within groove (226), first portion (216) is securely yet selectively attached with second portion (218) of pin holder assembly (200).

Another feature of pin holder assembly (200) includes an audible snap, click, or similar sound when first portion (216) is fully and securely attached with second portion (218) via the connection between connector (204) and adapter (206). This connection also provides for a tactile indication that first portion (216) is fully and securely attached with second portion (218). This tactile indication is at least in part due to the movement of resilient sphere portions (252) when seating within groove (226) of adapter (206).

As shown in the illustrated version, first and second pressure pins (248, 250) comprise resilient sphere portions (252). In some other versions resilient sphere portions (252), and/or first and second pressure pins (248, 250) partially or entirely, can be replaced with other similarly functioning structures as will be appreciated by those of ordinary skill in the art in view of the teachings herein.

Still referring to FIGS. 6 and 7, second portion (218) of pin holder assembly (200) comprises adapter (206) and actuator (208). Adapter (206) is attached with actuator (208) via threaded rod (268) and starburst connection as mentioned above. As shown, threaded rod (268) extends through disc (280) and starburst member (not shown) of actuator (208). A spring (not shown) is placed between starburst member (not shown) of actuator (208) and starburst member (272) of adapter (206). A more central threaded portion (269) of threaded rod (268) that extends distal of starburst member (not shown) of actuator (208) engages with threaded inner opening (264) of adapter (206). A distal threaded portion of threaded rod (268) that extends further distal of threaded inner opening (264) of adapter (206) passes through washer (256) and threadably engages with nut (258). Washer (256) and nut (258) are tightened such that they seat adjacent to annular surface (266) of adapter (206). Cap (260) then threadably engages threaded outer opening (262) of adapter (206) and covers nut (258). With this configuration, adapter (206) is securely attached to actuator (208) thereby making up second portion (218) of pin holder assembly (200).

As mentioned earlier, in use rotation of disc (280) of actuator (208) causes starburst member (not shown) of actuator (208) to translate within opening of second arm (14) such that starburst member (not shown) of actuator (208) moves from an engaged position with starburst member (272) of adapter (206) to a disengaged position with starburst member (272) of adapter (206) and vice versa. This translation is caused by pushing members (not shown) associated with disc (280) impinging a greater amount or a lesser amount on starburst member (not shown) of actuator (208) when disc (280) is rotated. The degree of impingement causes spring (not shown) to compress or expand accordingly. When starbursts of adapter (206) and actuator (208) are disengaged, connected first portion (216) and adapter (206) of pin holder assembly (200) are rotatable in unison about an axis defined by threaded rod (268). Once oriented in the desired position, starburst members of adapter (206) and actuator (208) can then be engaged to prevent rotation of pin holder assembly (200). Independent of this rotation functionality, first portion (216) of pin holder assembly (200) is separable from second portion (218) of pin holder assembly to allow for interchangeability of arch member (202) as shown and described above.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometries, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An interchangeable pin holder assembly for use with a stabilization device, wherein the interchangeable pin holder assembly comprises:
(a) a first member comprising:
(i) a groove, and
(ii) a first rotation limiting member separate from the groove; and
(b) a second member configured to connect with a pin holder, wherein the second member comprises:
(i) at least one resiliently biased member, wherein the at least one resiliently biased member is configured to selectively engage with the groove to prevent unintended separation of the first member from the second member when the at least one resiliently biased member is positioned within the groove, and
(ii) a second rotation limiting member separate from the at least one resiliently biased member, wherein the second rotation limiting member is configured to engage with the first rotation limiting member to prevent rotation of the first member relative to the second member when the second rotation limiting member is engaged with the first rotation limiting member.

2. The interchangeable pin holder assembly of claim 1, wherein the resiliently biased member of the second member comprises a resiliently biased sphere.

3. The interchangeable pin holder assembly of claim 2, wherein the groove of the first member comprises a chamfered edge.

4. The interchangeable pin holder assembly of claim 3, wherein the resiliently biased sphere is configured to selectively engage the chamfered edge of the groove of the first member.

5. The interchangeable pin holder assembly of claim 2, wherein the resiliently biased sphere is configured to selectively engage with the groove, wherein the resiliently biased sphere is configured to make an auditable noise when engaging the groove.

6. The interchangeable pin holder assembly of claim 1, wherein the second rotation limiting member comprises a protrusion, wherein the first rotation limiting member comprises a recess, wherein the recess is configured to receive the protrusion, wherein the recess and protrusion are sized to prevent substantially all independent rotational movement of the first member relative to the second member.

7. The interchangeable pin holder assembly of claim 1, wherein the first member comprises an extension, wherein the groove is positioned on the extension, and wherein the second member comprises an opening configured to receive the extension within the opening.

8. The interchangeable pin holder assembly of claim 7, wherein the extension of the first member and the opening of the second member are configured to selectively engage to restrict movement of the first member relative to the second member in a select one or more of a lateral direction and a vertical direction.

9. A method for interchanging a portion of a pin holder assembly of a stabilization device comprising the steps of:
(a) applying a force on a first removable portion of the pin holder assembly in a direction away from a fixed portion of the pin holder assembly configured to directly connect with the stabilization device, wherein the first removable portion of the pin holder assembly comprises at least one resiliently biased member and a first rotational lock member separate from the at least one resiliently biased member, wherein the fixed portion of the pin holder assembly comprises a recessed area and a second rotational lock member separate from the recessed area;
(b) disengaging the at least one resiliently biased member of the first removable portion from the recessed area of the fixed portion;
(c) disengaging the first rotational lock member of the first removable portion from the second rotational lock member of the fixed portion;
(d) aligning a second removable portion of the pin holder assembly with the fixed portion of the pin holder assembly; and
(e) applying a force on the second removable portion in a direction toward the fixed portion of the pin holder assembly.

10. The method of claim 9, wherein the at least one resiliently biased member comprises a resilient spherical portion, wherein the recessed area comprises a groove having a chamfered edge, wherein disengaging the at least one resiliently biased member from the recessed area causes the resilient spherical portion to move along the chamfered edge.

11. The method of claim 9, wherein the act of disengaging the at least one resiliently biased member from the recessed area and the act of disengaging the first rotational lock member from the second rotational lock member occurs substantially simultaneously.

12. The method of claim 9, wherein the second removable portion comprises at least one resiliently biased member, wherein applying a force on the second removable portion causes the at least one resiliently biased member of the second removable portion to engage the recessed area of the fixed portion.

13. The method of claim 12, wherein aligning of the second removable portion with the fixed portion permits the at least one resiliently biased member of the second removable portion and a first rotational lock member of the second removable portion to engage the recessed area and the second rotational lock member of the fixed portion when the force to the second removable portion is applied.

14. An interchangeable pin holder assembly for use with a stabilization device, wherein the interchangeable pin holder assembly comprises:
(a) a first member and a second member, wherein the first and second members are configured to eliminate a select one or both of lateral movement and vertical movement between a first portion of the interchangeable pin holder assembly and a second portion of the interchangeable pin holder assembly, when the first portion of the interchangeable pin holder assembly is selectively connected with the second portion of the interchangeable pin holder assembly; and
(b) a third member and a fourth member, wherein the third member and the fourth member are separate from the first member and the second member, wherein the third and fourth members are configured to eliminate rotational movement between the first portion of the interchangeable pin holder assembly and the second portion of the interchangeable pin holder assembly when the first portion of the interchangeable pin holder assembly is selectively connected with the second portion of the interchangeable pin holder assembly.

15. The interchangeable pin holder assembly of claim 14, wherein the first member comprises an extension, wherein the second member comprises an opening.

16. The interchangeable pin holder assembly of claim 14 further comprising a fifth member comprising one or more resiliently biased members, and a sixth member comprising a groove.

17. The interchangeable pin holder assembly of claim 16, wherein the one or more resiliently biased members are configured to allow the first portion of the interchangeable pin holder assembly to disengage from the second portion of the interchangeable pin holder assembly when the first portion of the interchangeable pin holder assembly is pulled away from the second portion of the interchangeable pin holder assembly with sufficient force to overcome bias of the resiliently biased members.

18. The interchangeable pin holder assembly of claim 14, wherein the third member and the fourth member are configured to engage one another.

19. The interchangeable pin holder assembly of claim 18, wherein the third member comprises a protrusion, wherein the fourth member comprises a recess, wherein the protrusion and the recess are sized such that the protrusion fits within the recess such that no rotational movement between the first portion and the second portion of the pin holder assembly is permitted when the protrusion is within the recess.

20. The interchangeable pin holder assembly of claim 14, wherein the first and fourth members are located on the first portion of the interchangeable pin holder assembly, wherein the second and third members are located on the second portion of the interchangeable pin holder assembly, wherein the spacing between the first member and the fourth member does not control restrictions on movement between the first portion and the second portion of the interchangeable pin holder assembly, and wherein the spacing between the second member and the third member does not control restrictions on movement between the first portion and the second portion of the interchangeable pin holder.
